(19) United States
(12) Patent Application Publication
(10) Pub. No.: US 2012/0030958 A1
(43) Pub. Date: Feb. 9, 2012

(54) PEDIATRICIAN'S EXAMINATION TABLE COVER

(76) Inventor: Michelle Mihalcik, Burlington, CT (US)

(21) Appl. No.: 12/849,303

(22) Filed: Aug. 3, 2010

(51) Int. Cl. G01B 3/10; A61B 5/107; G01B 1/00

(52) U.S. Cl. 33/701; 33/759

(57) ABSTRACT

A roll of paper dimensioned to cover the surface of an associated medical examination table. Successive axial sections of the free end of the roll are elongated axially. Each axial section includes a plurality of indicia representative of units of length disposed on the paper at axially spaced locations on the axial section. The indicia are elongated laterally with respect to the axial section. In some embodiments the respective indicia are differentiated by length or color. The respective indicia may be disposed at axial intervals along substantially the entire axial extent of the entire roll. In some embodiments the respective indicia extend laterally across a substantial part of the lateral extent of the axial section.

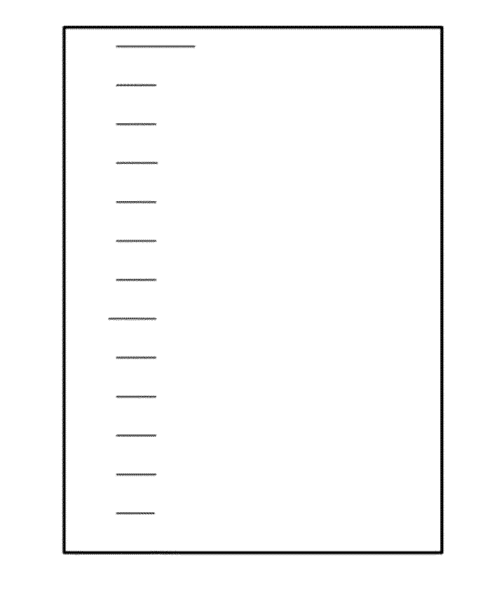

### FIELD OF THE INVENTION

The invention has particular application to methods and apparatus for measuring the height of infants. Pediatricians and other medical professionals routinely measure the height and weight of infants at chronological intervals.

### BACKGROUND OF THE INVENTION

The prior art includes the traditional technique of placing the infant on an examination table and utilizing a cloth tape measure for measuring the height of the infant. This technique can be performed by a single person despite the need to simultaneously secure the infant from moving, grasp and position the cloth tape measure along the length of the child, and read the height of the infant from the tape measure.

### SUMMARY OF THE INVENTION

It is an object of the present invention to simplify the process of measuring the height of an infant.

Another object of the present invention is to provide apparatus that is inexpensive to manufacturer and decreases the time required to measure the height of an infant.

It has now been found that these and other objects of the invention may be achieved in a roll of paper dimensioned to cover the surface of an associated medical examination table with successive axial sections of the free end, each axial section being elongated axially wherein each axial section includes a plurality of indicia representative of units of length disposed on the paper at axially spaced locations on the axial section, the indicia being elongated laterally with respect to the axial section.

In some embodiments the respective indicia are differentiated by length or color. The respective indicia may be disposed at axial intervals along substantially the entire axial extent of the entire roll. In some embodiments the respective indicia extend laterally across a substantial part of the lateral extent of the axial section.

In other forms of the present invention a discrete sheet of paper is dimensioned to cover the surface of an associated medical examination, each sheet is elongated and thereby defining an axial direction of the sheet wherein each sheet includes a plurality of indicia representative of units of length disposed on the paper at axially spaced locations on the sheet, the indicia being elongated laterally.

In these other forms of the present invention respective indicia are differentiated by length or color.

The respective indicia may be disposed at axial intervals along substantially the entire axial extent of the sheet. Similarly, when the respective indicia may extend laterally across a substantial part of the lateral extent of the sheet.

The invention also includes the method for facilitating the measurement of the height of an infant which includes providing a roll of paper having a width substantially equal to the width of an associated examination table; and printing indicia representative of linear units of measurement on successive axial parts of one face of the paper along substantially the entire axial extent of the paper on the roll whereby axial sections of the free end of the roll of paper when disposed on an examination table facilitate registration of an infant with the indicia to measure the height of the infant.

### BRIEF DESCRIPTION OF THE DRAWING

The invention will be better understood by reference to the accompanying drawing which diagrammatically illustrates an axial section of a roll of paper used to cover the examination table.

### DESCRIPTION OF THE PREFERRED EMBODIMENTS

Doctor's offices and hospitals have examination tables that typically have a roll of paper, often 125 or 230 long. For purposes of description, the axial part of the roll that is accessible to the user will be referred to as the free or loose end of the roll. In traditional use, the loose end of the roll of paper is drawn over the table to provide a sanitary examination work site for each new patient.

The drawing illustrates an axial section of the roll of paper in a preferred embodiment of the invention. In such embodiment the roll is provided with indicia disposed at intervals corresponding to the local linear measurement system. That system may be the English or Metric system or any other system. The drawing illustrates diagrammatically the indicia that may be printed on the entire axial extent of the roll. The indicia may, for example have a longer indicia for each foot and shorter indicia for inch intervals and even shorter indicia for fractional parts of each inch in the same manner as a ruler printed on the roll of paper with printing extending from the free end to the end of the paper that is typically on a core. Stated another way the rule extends the axial extent of the paper. Because the ruler is continuous, in a preferred embodiment, this means that when the nurse or doctor rips off the free end, the remaining free end is available for measuring an infant without the need to position the infant in registration with a fixed point.

In some embodiments the indicia will have no numeric markings on the paper adjacent to any indicia and the user will merely count individual indicia, such as indicia indicative of respective inches to measure the height of the infant. In some embodiments the length of the some of the respective inch markings may be distinctive. For example, the indicia for each successive 10 or 12 inch interval is distinctive in some embodiments of the present invention. For example, each 10th or 12^{th} inch indicia may be a unique color or a different length that differentiates those indicia from other inch indicia. Similarly, each 5th inch indicia (or any other periodic indicia) may be a unique color or a different length that differentiates those indicia from other inch indicia. Thus, the user may quickly measure an infant without having numerical values adjacent to respective indicia. Although this description refers to indicia identifying inch intervals, those skilled in the art will recognize that other embodiments may utilize a unique indicia for a particular periodic interval such as 5 cm.

Still other embodiments may have numerical values printed adjacent to respective indicia. Because of the continuous nature of a roll of paper this will not be as common. Embodiments of the present invention using discrete sheets of paper instead of a roll of paper will have numeric values adjacent respective indicia. Other embodiments may use differentiating colors to distinguish, for example, inch idicia from fractions of an inch indicia. Such embodiments may have indicia that extend laterally across a much larger portion of the axial portion of paper covering the table. Some embodiments may use discrete sheets for covering the table.

Although the description above contains many specifics, these should not be construed as limiting the scope of the invention, but as merely providing illustrations of some of the presently preferred embodiments of this invention. Thus, the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by the appended claims, in which reference to an element in the singular is not intended to mean “one and only one ” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A roll of paper dimensioned to cover the surface of an associated medical examination table with successive axial sections of the free end, each axial section being elongated axially wherein:
each axial section includes a plurality of indicia representative of units of length disposed on said paper at axially spaced locations on said axial section, said indicia being elongated laterally with respect to the axial section.

2. The apparatus as described in claim 1 wherein respective indicia are differentiated by length.

3. The apparatus as described in claim 1 wherein respective indicia are differentiated by color.

4. The apparatus as described in claim 1 wherein respective indicia are disposed at axial intervals along substantially the entire axial extent of the entire roll.

5. The apparatus as described in claim 1 wherein at least some of the respective indicia extend laterally across a substantial part of the lateral extent of the axial section.

6. A discrete sheet of paper dimensioned to cover the surface of an associated medical examination, each sheet being elongated and thereby defining an axial direction of the sheet wherein:
each sheet includes a plurality of indicia representative of units of length disposed on said paper at axially spaced locations on said sheet, said indicia being elongated laterally.

7. The apparatus as described in claim 2 wherein respective indicia are differentiated by length.

8. The apparatus as described in claim 6 wherein respective indicia are differentiated by color.

9. The apparatus as described in claim 6 wherein respective indicia are disposed at axial intervals along substantially the entire axial extent of the sheet.

10. The apparatus as described in claim 6 wherein at least some of the respective indicia extend laterally across a substantial part of the lateral extent of the sheet.

11. The method for facilitating the measurement of the height of an infant which comprises:
providing a roll of paper having a width substantially equal to the width of an associated examination table; and
printing indicia representative of linear units of measurement on successive axial parts of one face of the paper along substantially the entire axial extent of the paper on the roll whereby axial sections of the free end of the roll of paper when disposed on an examination table facilitate registration of an infant with the indicia to measure the height of the infant.
